(19) United States
(12) Patent Application Publication
(10) Pub. No.: US 2014/0051937 A1
(43) Pub. Date: Feb. 20, 2014

(54) THROAT SPRAY DEVICE WITH ILLUMINATION

(71) Applicant: Robert Joseph Baschnagel, III, Garden City, NY (US)

(72) Inventor: Robert Joseph Baschnagel, III, Garden City, NY (US)

(21) Appl. No.: 13/585,589

(22) Filed: Aug. 14, 2012

(51) Int. Cl. A61B 1/06; A61M 11/00

(52) U.S. Cl. 600/249

(57) ABSTRACT

A throat spray device with light apparatus, which providing lightening and allow the user of the throat spray to see his or her throat in a mirror, is disclosed in the present invention. Said throat spray device comprises of a throat spray of any kind that is commercially appropriate, a light apparatus with attachment means which attach the light apparatus to said throat spray. Said light apparatus is powered with a battery and has lightening means controlled by a on/off switch.

### BACKGROUND OF THE INVENTION

1. Field of the Invention

The present invention relates to a throat spray device. More particularly, the present invention relates to a throat spray device with a light apparatus.

Throat sprays are commonly used delivery methods of various medications for treatments of many kinds of symptoms and diseases. When using a throat spray, a user usually positions the throat spray tube or nozzle, which is the end where the medical compositions come through, inside the user's mouth and towards to the user's throat. Without the aid of a mirror the user will merely spry the medication in the general direction of the sore area of the back of the throat. This approach usually leads to an overall coating of the medication within the mouth, on the tongue and maybe or maybe not on the sore target area of the back of the throat itself. For such medications to be effective, they usually need to hit the target soar area and for the comfort of the user the sore area alone. This is because most throat medications have a numbing effect associated with them and by applying the spray to the general mouth area can lead to discomfort and numbing of the tongue and a lack of relief of throat pain itself.

Very often, a mirror is used by a spray user to help him or her better position the throat spray tube or nozzle towards to his or her throat. The problem with this approach is that the mirror alone is not enough for an individual to see the dark back area of one's own mouth—this being the case both under the aid of natural light or under the aid of indoor light above a wall mirror within a bathroom setting. Again, and as a result, it is very often the case that the medical compositions within the spray bottle itself are sprayed on the user's tongue or other parts of the user's mouth diminishing its intended use and effect.

Therefore, it is desirable to have a throat spray device with a light apparatus near the spry nozzle itself which can enable the user to see his or her throat in a mirror under the light by the light apparatus attached to the front end of the throat spray and effectively target the medicated spry onto the red or soar area of the back of the throat.

2. Description of Related Art

There exists various spray bottles within the marketplace today but none are with an lighting apparatus that is removable and attached to the head of the spray bottle itself as an aid for viewing a sore spot in ones throat so as to be able to exactly spray a soothing medicated fluid thereon.

For example, U.S. Pat. No. 6,345,774 to Calhoun discloses a spray bottle spray head for spraying a fluid there from over a large area. The spray bottle spray head includes a bottle with a pump body coupled thereto. A generally frusta-conical spray head is coupled to the pump body. The spray head has a generally circular front face and tapers from the front face of the spray head to the pump body. The front face of the spray head has a plurality of spaced apart spray apertures therein.

In another example, U.S. Pat. No. 6,206,243 to Schneider & McLean discloses a spray bottle for holding and dispensing a liquid comprises a collapsible container for holding the liquid, a spray head for dispensing and atomizing the liquid, and an elongated coupling for securing the spray head to the container and providing a handhold. The container is adapted to be collapsed into a substantially flat panel when empty and to be distended when the bottle is filled with the liquid. With the spray bottles empty and the containers collapsed, a retail display may comprise a plurality of spray bottles. The spray bottles are arranged in at least one row with the containers collapsed such that each spray head is in an abutting relationship with the spray head of at least one adjacent spray bottle.

There appears to be a need for an illuminated spray bottle that will address the issue of visually aiding an individual when applying throat spray to the back of one's mouth.

### SUMMARY OF THE INVENTION

In an exemplary embodiment of the present invention, there is disclosed a throat spray device, and more specifically, a throat spray device with a light apparatus which provides lightening and allows the user of the throat spray to see his or her throat in a mirror. In a particular embodiment of the present invention, said throat spray device is comprised of a throat spray of any kind that is commercially appropriate, a light apparatus with attachment means which attaches the light apparatus to said throat spray. Said light apparatus is powered with a battery or batteries and has lightening means controlled by an on/off switch attached therein. Said light apparatus can either be geminately attached or temporarily attached to a spray nozzle and is of uniform shape in regard to the attachment section to as be able to attach to generic bottle spray nozzles.

The embodiment described herein is of a small size and does not greatly increase the size of the spray nozzle upon which it is attached. It is able to easily fit within ones mouth and be positioned towards the far back therein.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a perspective view of the present invention of a throat spray device with light apparatus.

FIG. 2 is a perspective view of said light apparatus of the present invention.

FIG. 3 is the rear view of the said light apparatus of the present invention.

FIG. 4 is the cross-section view of said light apparatus of the present invention attached to a throat spray tube/nozzle.

### DESCRIPTION OF THE PREFERRED EMBODIMENT

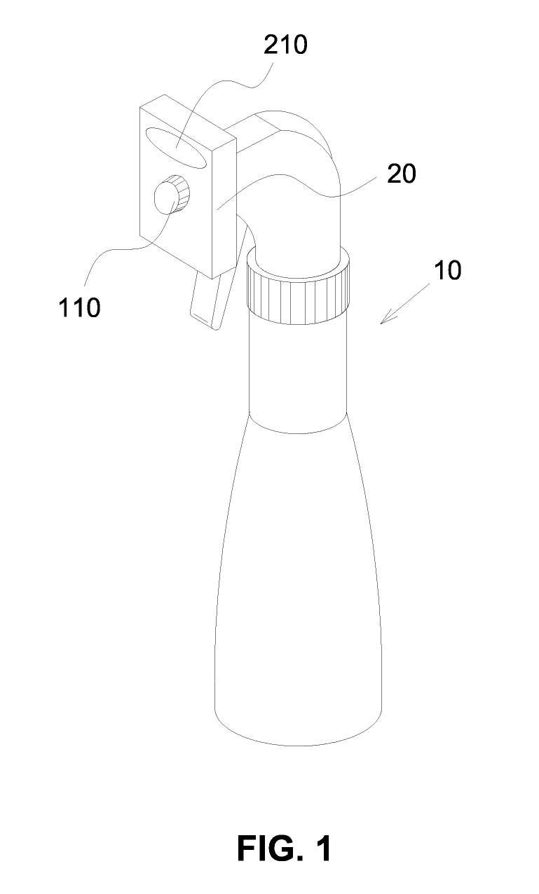

Referring to FIG. 1, there is disclosed a throat spray device 1 comprising of a throat spray 10 of any kind that is commercially appropriate, and a light apparatus 20. Said throat spray 10 includes a throat spay tube/nozzle 110 which is the end part where medical compositions come through. Said light apparatus includes at least one lightening means 210 which provides enough lightening and allows the user of the throat spray device see his or her throat in a mirror.

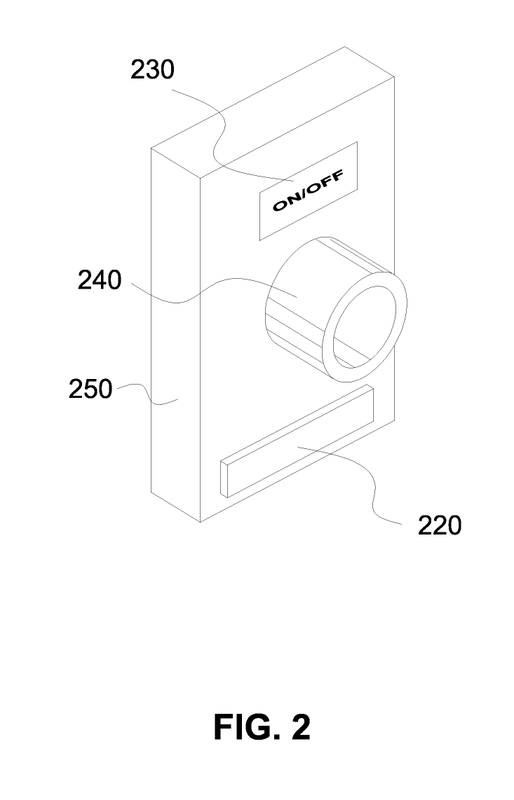

As shown in FIG. 2, said light apparatus 20 further includes a electric power supply source 220, an on/off switch 230, an attaching means 240 which attaches said light apparatus 20 to the throat spray tube/nozzle 110, and a housing device 250 which commentates 210, 220, 230, 240 and 250.

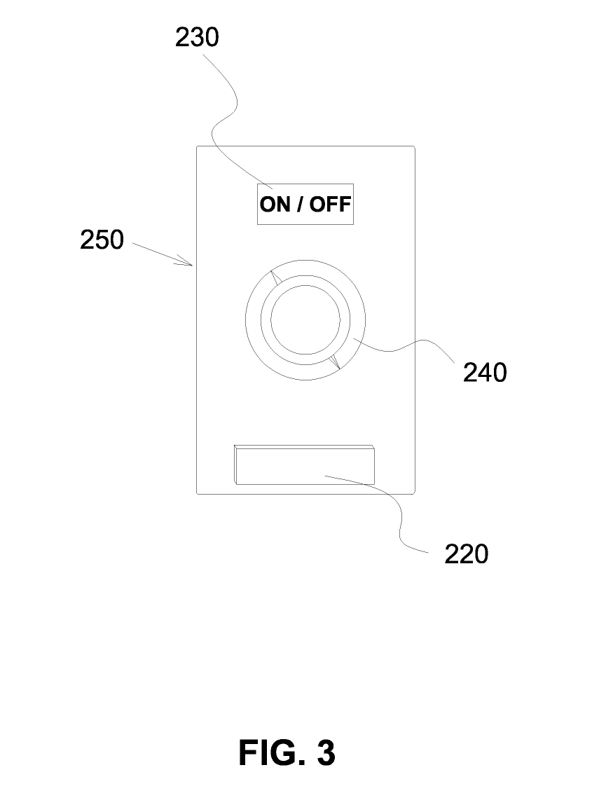

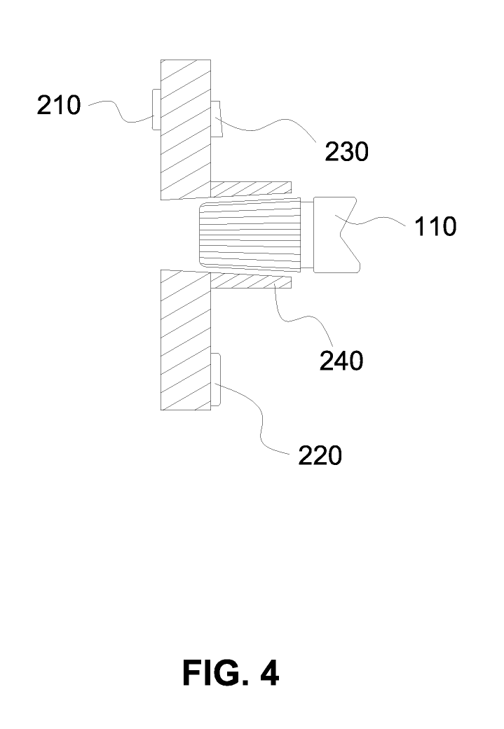

In one preferred embodiment, as shown in FIG. 3 and 4, the lightening means 210 is located on the front side of said light apparatus 20, towards to the user's throat. Said on/off switch 230 and power source 220 are located on the back side of said light apparatus 20, towards to said throat spray 10.

In another embodiment of present invention, said light apparatus is pre-attached to the throat spray tube/nozzle of said throat spray.

In another embodiment of present invention, said light apparatus is not pre-attached to the throat spray, but is able to be attached to the throat spray tube/nozzle of said throat spay.

In another embodiment of present invention, said electric power supply source is a battery.

In another embodiment of present invention, said lightening means includes, but not limited to, light bulbs, LED lights or other suitable light sources.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

While various embodiments have been described and pictorially depicted herein, it should be understood that they have been presented by way of example only, and not of limitation. Likewise, the various figures depict an example of the preferred embodiment, which is done to aid in understanding the features and functionality that are included in the embodiment. The present system is not restricted to the illustrated example designs or configurations, but said features may be implemented using a variety of alternative designs and configurations.

What is claimed is:

1. A throat spray device with light apparatus, comprising of a throat spray of any kind that is commercially appropriate and a light apparatus that is removably attached.

2. A throat spray device of claim 1, wherein said throat spray has a tube/nozzle which is the end part where medical compositions may come through.

3. A throat spray device of claim 2, wherein said light apparatus is pre-attached to said tube/nozzle of said throat spray, or separated from said throat spay but is able to be attached to said tube/nozzle of throat spray.

4. A throat spray device of claim 3, wherein said light apparatus further comprising of a lightening means which provides enough lightening and allows the user of the throat spray device see his or her throat in a mirror, an electric power supply source, an on/off switch, an attaching means and a house device which holds said lightening means, power supply source, on/off switch and attaching means.

5. A throat spray device of claim 4, wherein said lightening means includes, but not limited to, light bulb, LED lights, or other similar light sources.

6. A throat spray device of claim 4, wherein said electric power supply source is a battery.

7. A throat spray device of claim 4, wherein said attaching means includes, not limited to screwing ,clipping or any other commonly used attaching methods.

8. A throat spray device of claim 4, wherein said lightening means includes light bulbs or LED lights or other similar light sources, said electric power supply source is a battery, and said attaching means includes screwing, clipping or other attaching methods.
